(19) United States
(12) Patent Application Publication
(10) Pub. No.: US 2015/0147799 A1
(43) Pub. Date: May 28, 2015

(54) HALOGEN-FREE HIGH-FREQUENCY RESIN COMPOSITION

(71) Applicants: ITEQ CORPORATION, Ping Chen (TW); ITEQ (DONGGUAN) CORPORATION, Dongguan City (CN)

(72) Inventors: Hailin Li, Dongguan City (CN); Faquan Tu, Dongguan City (CN); Tsung-Lieh Weng, Dongguan City (CN); Yongxin Jiang, Dongguan City (CN); Feng Tang, Dongguan City (CN); Quansheng Zhu, Dongguan City (CN)

(21) Appl. No.: 14/088,499

(22) Filed: Nov. 25, 2013

(51) Int. Cl. C08L 63/00; C12N 9/16; H05K 1/03; C08L 25/08; C08L 71/00

(52) U.S. Cl. CPC C08L 63/00; C08L 25/08; C08L 71/00; H05K 1/0373; C12N 9/16
USPC 435/196; 525/117; 524/508

(57) ABSTRACT

Disclosed is a halogen-free high-frequency resin composition calculated according to parts by weight, and including 20-50 parts by weight of dicyclopentadiene epoxy resin, 10-40 parts by weight of styrene-maleic anhydride copolymer, 10-30 parts by weight of benzoxazine resin, 5-20 parts by weight of polyfunctional epoxy resin and 20-40 parts by weight of at least one phosphorus-containing flame retardant. A copper clad laminate made of the halogen-free high-frequency resin composition has excellent properties including a low dielectric constant, a low dielectric loss, a high heat resistance, a low water absorption, a low coefficient of expansion and a high PCB manufacturability.

### FIELD OF THE INVENTION

The present invention relates to a halogen-free high-frequency resin composition.

### BACKGROUND OF THE INVENTION

The RoHs and WEE directives on the restriction and prohibition of the use of certain hazardous substances in electrical and electronic equipment were adopted by the European Union in February 2003, and the former relates to the directive of restricting and prohibiting the use of certain toxic, hazardous substances and elements of electrical and electronic equipments and the later relates to the directive of recycling waste electrical and electronic equipments. The WEEE directive took effect on August 2005 and the RoHs directive took effect on July 2006. To pass new standards of this sort, the use of traditional halogen-containing flame retardant materials should be reduced slowly until they are eliminated. In addition, the combustion of halogen-containing flame retardants or resins produces a large quantity of smoke and toxic and corrosive gases, which jeopardize human body and environment substantially. In particular, the European Union restricts the application of halogen flame retardants in electronic and circuit industries by laws, so that it is imperative to develop halogen-free green clad copper laminates.

After the aforementioned two European Union's directives were promulgated, printed circuit board manufacturers also request clad copper laminate manufacturers to develop halogen-free green clad copper laminate substrates. At present, the electronic industry blooms, and the performance requirements of clad copper laminates becomes increasingly higher, particularly for three major portable electronic products, satellite transmission and communication electronic products. The factors affecting the performance of the aforementioned products basically include the dielectric coefficient (Dk) and the dielectric loss tangent (Df) of the substrate. The smaller the dielectric coefficient of the substrate, the faster the signal transmission rate, the smaller the dielectric loss tangent value, the more complete the signal transmission, and the higher the signal authenticity. Particularly, present electronic products are developed with a light, thin and compact design and an increasingly higher transmission rate (over 1 GHz), and it is a main subject for related manufacturers to develop high-performance halogen-free high-frequency printed circuit board.

On the other hand, the conventional lead-free high-frequency printed circuit board generally uses bromine for flame retardation, but carbon-bromide (C—Br) bonds with low bond energy may be broken easily at high temperature, and thus causing the delamination of the substrate. Therefore, insufficient heat resistance becomes a major issue in the manufacture of circuit boards. Particularly, the present high-density interconnects (HDI) technology has increasingly higher requirements, and the issue of insufficient heat resistance limits the development of the HDI technology, particularly the high-frequency HDI technology. In addition, the present electronic products require the properties of high density and high reliability, and thus the substrate must have excellent hear resistance, low coefficient of expansion, chemical resistance, and dimension stability, so that the development of high-frequency printed circuit boards with high heat resistance and low coefficient of expansion becomes a trend of developing high-frequency substrates.

### SUMMARY OF THE INVENTION

In view of the aforementioned shortcomings of the prior art, it is a primary objective of the present invention to overcome the shortcomings by providing a halogen-free high-frequency resin composition, so that the manufactured clad copper laminate features the advantages of lower dielectric constant and dielectric loss, excellent heat resistance, good manufacturability and low coefficient of expansion and meets the halogen-free environmental protection requirements.

To achieve the aforementioned objective, the present invention provides a halogen-free high-frequency resin composition, comprising: 20-50 parts by weight of a dicyclopentadiene epoxy resin; 10-40 parts by weight of a styrene-maleic anhydride copolymer; 10-30 parts by weight of a benzoxazine resin; 20-40 parts by weight of at least one phosphorus-containing flame retardant; and 5-20 parts by weight of a polyfunctional epoxy resin; and the molecular structural formula of the dicyclopentadiene epoxy resin is shown below:

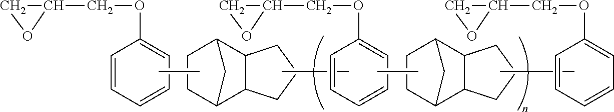

The molecular structural formula of the styrene-maleic anhydride copolymer is shown below:

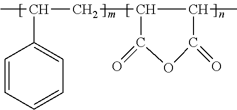

Where, m:n=3:1.

The benzoxazine resin is one or more resin selected from the group consisting of a bisphenol-A benzoxazine resin, a bisphenol-F benzoxazine resin, and a phenolphthalein benzoxazine resin.

The phosphorus-containing flame retardant includes one or more compounds selected from the group consisting of a phosphatase, a phosphazene compound, a phosphaphenanthrene and a derivative thereof.

The polyfunctional epoxy resin includes one or more epoxy resins selected from the group consisting of a trifunctional epoxy resin, a biphenyl epoxy resin, and a naphthalene ring epoxy resin.

Compared with the prior art, the present invention has the following advantages and effects:

1. The use of the dicyclopentadiene epoxy resin is capable of providing a lower dielectric constant, and the existence of dicyclopentadiene can provide excellent heat resistance and manufacturability for circuit boards.

2. The styrene-maleic anhydride copolymer having the anhydride structure can react with epoxy resin and also has the benzene ring structure capable of providing the properties of high heat resistance and low water absorption rate. Particularly a three-dimensional interpenetrating network is formed after the reaction to provide a lower dielectric loss value of the material.

3. The use of the benzoxazine resin with a specific flame retardation effect can assist the phosphorus-containing flame retardant for the flame retardation and reduce the consumption of the phosphorus-containing flame retardant (since the phosphorus-containing flame retardant absorbs moisture easily, so that the substrate may be delaminated easily), so as to reduce the water absorption rate and the risk of delamination. In addition, the resin of this type further has a good dielectric performance and its cured product has a good PCB manufacturability.

4. The composition of the present invention has a polyfunctional epoxy resin capable of reducing the coefficient of expansion of the substrate significantly and improving the manufacturability and reliability of the substrate.

5. The laminates made of this resin composition has the properties of low dielectric constant, low dielectric loss value, high heat resistance, and low water absorption to overcome the shortcomings including the poor heat resistance, high water absorption rate, and poor PCB manufacturability of the conventional high-frequency clad copper laminate, so that the laminates can have good applications in multi-layer boards.

6. Inorganic materials are added to lower the cost, and the inorganic filler such as silicon dioxide can reduce the coefficient of expansion and improve the heat resistance and flame retardation effects.

### BRIEF DESCRIPTION OF THE DRAWINGS

None

### DESCRIPTION OF THE PREFERRED EMBODIMENTS

The aforementioned and other objectives and advantages of the present invention will become clearer in light of the following detailed description of an illustrative embodiment of this invention described in connection with the drawings. It is intended that the embodiments and drawings disclosed herein are to be considered illustrative rather than restrictive.

The aforementioned properties are described by the following embodiments and examples of a control group, and Embodiments 1-7 and Examples of a control group 1-3 are described below.

The proportion of related substances divided into organic matters, respectively A1, A2, A3, B1, B2, C1 and C2, D calculated according to 100 parts by weight, and the percentage occupied by other compositions is the total weight percentage of the organic matters.

(A1) Styrene-maleic anhydride copolymer, SMA-EF30 (m:n=3:1)

(A2) Phenolphthalein benzoxazine

(A3) Bisphenol A benzoxazine

(B1) Dicyclopentadiene (modified DCPD) epoxy resin

(B2) Trifunctional epoxy resin

(C1) Phosphorus-containing phenolic resin

(C2) Phosphatase

(D) Melted silica power

The molecular structural formula of the trifunctional epoxy resin is shown below:

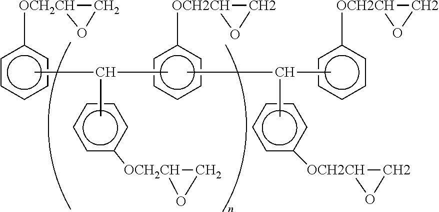

The laminate substrate of the present invention is produced by the aforementioned halogen-free high-frequency resin composition which is melted, dipped, glued, heated, and laminated. In the gluing process, a fiberglass cloth with the 2116 specification, a lamination specification of 2116*6 ply, and a thickness approximately equal to 0.8 mm. In addition, a copper foil with a thickness of 35 um (or a weight of 1 oz) is used for the lamination, and the copper foil is produced and pressed by a hot press machine, and the temperature of the material is controlled above and maintained for 100 min.

Recipe of Composition (1) calculated according to parts by weight

| | Embodi- | Embodi- | Embodi- | Embodi- | Embodi- |
|---|---|---|---|---|---|
| | ment 1 | ment 2 | ment 3 | ment 4 | ment 5 |
| A1 | 20 | 20 | 15 | 20 | 10 |
| A2 | 18 | | 18 | 18 | 28 |
| A3 | | 18 | | | |
| B1 | 30 | 30 | 40 | 25 | 30 |
| B2 | 10 | 10 | 5 | 15 | 10 |
| C1 | 22 | 22 | 22 | | 22 |
| C2 | | | | 22 | |
| D | 25 | 25 | 25 | 25 | 25 |

Recipes of the Composition calculated
according to parts by weight Table (2)

| | Embodi- | Embodi- | Embodi- | Example 1 | Example 2 |
|---|---|---|---|---|---|
| | ment 6 | ment 7 | ment 8 | of Control | of Control |
| A1 | 36 | 14 | 15 | | 35 |
| A2 | 11 | 12 | 18 | 30 | 30 |
| A3 | | | 5 | | |
| B1 | 26 | 26 | 30 | 35 | |
| B2 | 6 | 10 | 10 | 10 | 10 |
| C1 | 21 | 28 | 22 | 25 | 25 |
| C2 | | 10 | | | |
| D | 25 | 25 | 25 | 25 | 25 |

Recipe Performance Evaluation Table (1)

| Condition | | Embodiment 1 | Embodiment 2 | Embodiment 3 | Embodiment 4 | Embodiment 5 |
|---|---|---|---|---|---|---|
| Glass transition | ° C. | 194 | 170 | 175 | 155 | 186 |
| temperature | | | | | | |
| (Tg, ° C., DSC) | | | | | | |
| Thermal | min | >60 | >60 | >60 | >60 | >60 |
| stratification time | | | | | | |
| T-288° CTMA | | | | | | |
| (containing | | | | | | |
| cupper) | | | | | | |
| Peeling Strength | N/mm | 1.2 | 1.2 | 1.2 | 1.1 | 1.2 |
| (1 oz) | | | | | | |
| Water absorption | % | 0.37 | 0.41 | 0.37 | 0.35 | 0.38 |
| (PCT1h) | | | | | | |
| PCT 1h + dip (Dip) | min | >10 | >10 | >10 | >10 | >10 |
| Coppery clad | min | >30 | >30 | >30 | >30 | >30 |
| floating solder | | | | | | |
| coefficient of | % | 2.5 | 2.8 | 2.8 | 2.6 | 2.5 |
| thermal | | | | | | |
| expansion Z-axis | | | | | | |
| CTE (%) | | | | | | |
| Flame retardation | UL94 | V-0 | V-0 | V-0 | V-0 | V-0 |
| dielectric constant | 1 GHz | 3.85 | 3.78 | 3.71 | 3.83 | 3.86 |
| Dielectric loss | 1 GHz | 0.0055 | 0.0054 | 0.0059 | 0.0048 | 0.0062 |
| value | | | | | | |
| Halogen content | % | 0.03 | 0.03 | 0.03 | 0.03 | 0.03 |

Recipe Performance Evaluation Table (2)

| | | Embodiment | Embodiment | Embodiment | Example 1 | Example 2 |
|---|---|---|---|---|---|---|
| Condition | | 6 | 7 | 8 | of Control | of Control |
| Glass transition | ° C. | 179 | 180 | 180 | 191 | 201 |
| temperature | | | | | | |
| (Tg, ° C.) | | | | | | |
| Thermal layer | min | >60 | >60 | >60 | 45 | 40 |
| division time | | | | | | |
| T-288° CTMA | | | | | | |
| (containing copper) | | | | | | |
| Peeling | N/mm | 1.2 | 1.2 | 1.2 | 1.4 | 1.4 |
| strength(1 oz) | | | | | | |
| Water absorption | % | 0.38 | 0.42 | 0.42 | 0.48 | 0.52 |
| PCT 1h + Dip | min | >10 | >10 | >10 | >10 | >10 |
| Copper clad | min | >30 | >30 | >30 | 18 | 15 |
| floating solder | | | | | | |
| Coefficient of | % | 2.4 | 2.7 | 2.7 | 3.1 | 3.3 |
| thermal expansion | | | | | | |
| Z-axis CTE(%) | | | | | | |
| Flame retardation | UL94 | V-0 | V-0 | V-0 | V-1 | V-1 |
| Dielectric constant | 1 GHz | 3.89 | 3.83 | 3.82 | 4.15 | 4.30 |
| Dielectric loss | 1 GHz | 0.0045 | 0.0063 | 0.0060 | 0.0092 | 0.0095 |
| constant | | | | | | |
| Halogen content | % | 0.03 | 0.03 | 0.03 | 0.03 | 0.03 |

The testing methods of the aforementioned properties are described below:

(1) Water absorption percentage: It is a percentage of the weight difference before and after the PCT steaming process with respect to the sample weight before the PCT takes place.

(2) Thermal layer division time: The delamination layer division time is recorded, after the PCT is steamed for an hour at 121° C. in 105 KPa pressure cooker, and dipped in the solder pot at 288° C.

(3) Copper clad floating solder: The delamination time is measured when the solder (at 288° C.) of a copper clad laminate floats on a solder pot.

(4) Thermal layer division time T-288: It is measured according to the IPC-TM-650 2.4.24.1 method.

(5) Coefficient of thermal expansion Z-axis CTE (TMA): It is measure according to the IPC-TM-650 2.4.24 method.

(6) Glass transition temperature (Tg): It is measured according to the differential scanning calorimetry (DSC) and the DSC method as set forth by the IPC-TM-6502.4.25 regulation.

(7) Dielectric constant and dielectric loss value: Both dielectric constant and dielectric loss value are measured below GHz by a parallel board method according to the IPC-TM-6502.5.5.9 regulation.

(8) Peeling strength: It is measured according to the IPC-TM-650 2.4.9 regulation.

(9) Combustibility: It is measured by a vertical combustion method according to the UL 94 regulation.

According to the aforementioned results, the laminates produced by the composition of the present invention feature low dielectric constant, low dielectric loss, low coefficient of expansion, high heat resistance, low water absorption, and refractory function, while providing excellent manufacturability. Further, the halogen content is less than 0.09%, thus achieving halogen-free flame retardations and meeting environmental protection requirements. In addition, the printed circuit boards produced by the composition of the present invention feature high heat resistance, excellent high-frequency dielectric property, and capability of meeting the increasingly higher requirement of the printed circuit boards for high-frequency transmission systems.

While the invention has been described by means of specific embodiments, numerous modifications and variations could be made thereto by those skilled in the art without departing from the scope and spirit of the invention set forth in the claims.

What is claimed is:

1. A halogen-free high-frequency resin composition, comprising:
20-50 parts by weight of a dicyclopentadiene epoxy resin;
10-40 parts by weight of a styrene-maleic anhydride copolymer;
10-30 parts by weight of a benzoxazine resin;
20-40 parts by weight of at least one phosphorus-containing flame retardant; and
5-20 parts by weight of a polyfunctional epoxy resin;
wherein, the molecular structural formula of the dicyclopentadiene epoxy resin is: 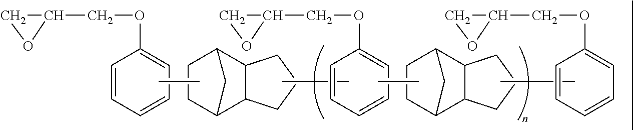
and the molecular structural formula of the styrene-maleic anhydride copolymer is: 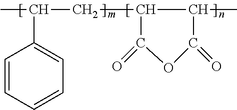
and m:n=3:1.

2. The halogen-free high-frequency resin composition of claim 1, wherein the benzoxazine resin includes one or more resins selected from the group consisting of a bisphenol-A benzoxazine resin, a bisphenol-F benzoxazine resin, and a phenolphthalein benzoxazine resin.

3. The halogen-free high-frequency resin composition of claim 1, wherein the phosphorus-containing flame retardant includes one or more compounds selected from the group consisting of a phosphatase, a phosphazene compound, a phosphaphenanthrene and a derivative thereof.

4. The halogen-free high-frequency resin composition of claim 1, wherein the polyfunctional epoxy resin includes one or more epoxy resins selected from the group consisting of a trifunctional epoxy resin, a biphenyl epoxy resin, and a naphthalene ring epoxy resin.

5. The halogen-free high-frequency resin composition of claim 1, further comprising one or more organic fillers selected from the group consisting of crystalline silica, melted silica, spherical silica, kaolin and talcum powder.
